(19) United States
(12) Patent Application Publication
(10) Pub. No.: US 2009/0108066 A1
(43) Pub. Date: Apr. 30, 2009

(54) OPTICAL SYSTEM FOR BARCODE SCANNER

(75) Inventor: Kai-Yuan Tien, Hsichih City (TW)

Correspondence Address: RIOTEC CO., LTD., P.O. BOX 108-00403, TAIPEI, TW

(73) Assignee: RIOTEC CO., LTD., Taipei City (TW)

(21) Appl. No.: 12/345,678

(22) Filed: Dec. 30, 2008

Related U.S. Application Data
(63) Continuation-in-part of application No. 11/423,955, filed on Jun. 14, 2006.

(51) Int. Cl. G06K 7/10

(52) U.S. Cl. 235/462.01

(57) ABSTRACT

An optical system used in a barcode scanner is disclosed to include a standing cylinder lens, a light source that emits a light beam through the standing cylinder lens onto the barcode of the product to be scanned, a focusing lens that focuses the image reflected by the barcode, a linear sensor array, and a slanted arranged reflector that reflects the image focused by the focusing lens onto the linear sensor array for reading. The light path of the image focused by the focusing lens onto the reflector and the light path of the image reflected by the reflector onto the linear sensor array are overlapped, forming a light path overlap region.

This application is a Continuation-In-Part of my patent application, Ser. No. 11/423,955, filed on Jun. 14, 2006.

### BACKGROUND OF THE INVENTION

1. Field of the Invention

The present invention relates to a barcode scanner and more particularly, to an optical system for barcode scanner, which has the component parts thereof arranged on one same plane in such a manner that the light path of the image focused by the focusing lens onto the reflector and the light path of the image reflected by the reflector onto the linear sensor array are overlapped, enabling the dimension of the barcode scanner to be minimized.

2. Description of the Related Art

Conventional barcode scanners commonly use a linear sensor array to receive reflective image from the barcode. The light beam on the barcode for the reflective image received by the linear sensor array is preferably in a uniform status to facilitate further barcode signal processing. In a conventional barcode scanner, the focal distance of the focusing lens is designed subject to the length from focusing lens to the linear sensor array so that the linear sensor array can effectively receive the reflective image of the barcode. Therefore, a wide space area is needed for the light path without any obstruction. In consequence, the dimension of the barcode scanner cannot be minimized. In order to eliminate this problem, an optical system for barcode scanner shown in FIG. 7 is created. According to this design, a reflector A1 is provided to reflect focused image from the focusing lens A2 onto the linear sensor array A, shortening the light path in x-axis direction. However, because the focal distance of the focusing lens A2 does not change, the distance X1 between the reflector A1 and the linear sensor array A plus the distance X2 between focusing lens A2 and the reflector A1 is equal to the focal distance X of the focusing lens A2. However, because the light path of the image reflected by the reflector is an independent light path, it requires an extra space in z-axis. Therefore, this design cannot minimize the installation space. Further, a preservation space B1 is provided beyond the light path space B of the linear sensor array A, reflector A1 and focusing lens A2 for the design of an illumination light source. However, this preservation space B1 is too small for direct use. Therefore, an extra space at one lateral side relative to the optical system is usually used for the installation of the illumination light source, increasing the dimension and manufacturing cost of the barcode scanner.

Therefore, it is desirable to provide an optical system for barcode scanner that eliminates the aforesaid problems.

### SUMMARY OF THE INVENTION

The present invention has been accomplished under the circumstances in view. It is one object of the present invention to provide an optical system for barcode scanner, which requires less installation space. It is another object of the present invention to provide an optical system for barcode scanner, which enhances uniformity of the light emitted by the light source, avoiding image noise and improving barcode reading reliability.

To achieve these and other objects of the present invention, an optical system for barcode scanner comprises a standing cylinder lens, a light source that emits a light beam through the standing cylinder lens onto the barcode of the product to be scanned, a focusing lens that focuses the image reflected by the barcode, a linear sensor array, and a slanted arranged reflector that reflects the image focused by the focusing lens onto the linear sensor array for reading. The light path of the image focused by the focusing lens onto the reflector and the light path of the image reflected by the reflector onto the linear sensor array are overlapped, forming a light path overlap region. By means of the characteristic of the light path overlap region, the optical system requires less installation space, and therefore the dimension of the barcode scanner can be greatly reduced.

Further, slight oscillation means is provided to cause slight oscillation of the emitted light beam in direction perpendicular to the light path of the light source, enhancing uniformity of the light, and therefore the reading reliability of the barcode scanner is greatly improved.

### BRIEF DESCRIPTION OF THE DRAWINGS

FIG. 1 is a schematic top view of an optical system for barcode scanner in accordance with the present invention.

FIG. 2 is a schematic side view of the optical system according to the present invention.

FIG. 3 is a schematic drawing showing one arrangement of the slight oscillation means according to the present invention.

FIG. 4 is a schematic drawing showing an alternate form of the slight oscillation means according to the present invention.

FIG. 5 is a schematic drawing showing another alternate form of the slight oscillation means according to the present invention.

FIG. 6 is a schematic drawing showing still another alternate form of the slight oscillation means according to the present invention.

FIG. 7 is a schematic side view of an optical system for barcode scanner according to the prior art.

### DETAILED DESCRIPTION OF THE PREFERRED EMBODIMENT

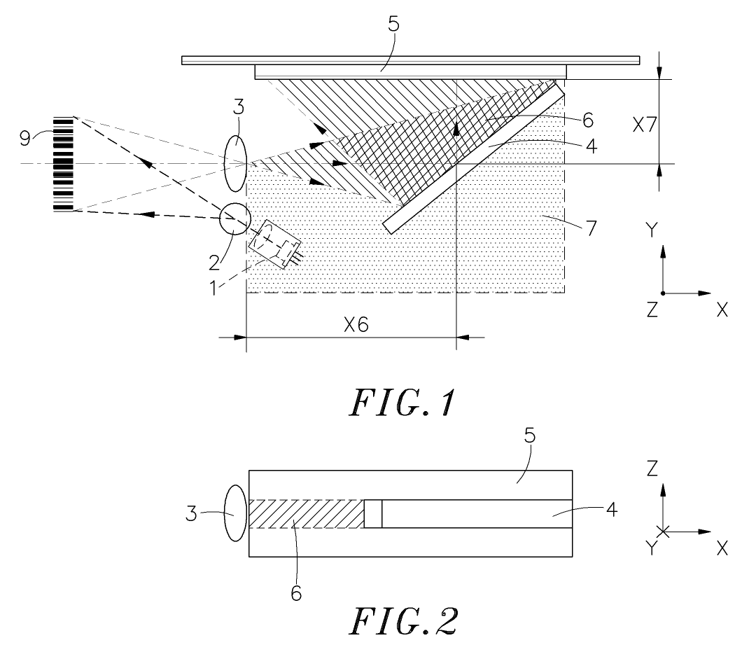

Referring to FIGS. 1 and 2, an optical system for barcode scanner in accordance with a first embodiment of the present invention is shown comprising a light source 1, a standing cylinder lens 2, a focusing lens 3, a reflector 4, and a linear sensor array 5.

The light source 1 is formed of a light emitting device and a collimator. Further, the light beam produced by the light source 1 falls upon the plane of the product's barcode 9 to be scanned at a slanting angle.

The standing cylinder lens 2 is adapted to expand the light beam produced by the light source 1 into a line-shaped light beam to fall upon the barcode 9 of the product to be scanned.

The focusing lens 3 is adapted to receive the barcode image reflected by the barcode 9 and to focus the barcode image. Further, the focal distance of the focusing lens 3 is X.

The reflector 4 is adapted to reflect the image focused by the focusing lens 3.

The linear sensor array 5 is adapted to receive the image focused by the focusing lens 3 and reflected by the reflector 4.

According to the aforesaid optical system, the standing cylinder lens 2 is disposed in the light path of the light source 1; the focusing lens 3 is disposed at one lateral side relative to the light source 1 and the standing cylinder lens 2; the reflector 4 is obliquely disposed at a rear side in the receiving light path of the focusing lens 3; a linear sensor array 5 is disposed at one lateral side relative to the focusing lens 3 and the reflector 4 on the same horizontal plane. The reflector 4 reflects the image that is focused by the focusing lens 3 onto the linear sensor array 5. Further, the light source 1, the standing cylinder lens 2, the focusing lens 3, the reflector 4 and the linear sensor array 5 are arranged on one same horizontal plane, providing a light path overlap region 6. This design lowers the height of the arrangement of the component parts, providing a low-profile characteristic.

During operation, the reflector 4 reflects the image of the barcode 9 focused by the focusing lens 3 onto the linear sensor array 5, wherein the light path X6 between the focusing lens 3 and the reflector 4 plus the light path X7 between the reflector 4 and the linear sensor array 5 is equal to the focal distance X of the focusing lens 3. However, because the light path of the focusing lens 3 overlaps with the light path of the light path of the reflector 4, both light paths form a light path overlap region 6 that diminishes surface occupation on x-y plane while surface occupation on x-z plane remains unchanged. The greater the overlap region is the smaller the occupation of the surface area will be. Further, a preservation region 7 is maintained intact for installation of illumination light source. By means of the characteristic of the light path overlap region 6, the dimension of the barcode scanner is greatly reduced, and therefore the invention eliminates the common drawback of big size of conventional barcode scanners.

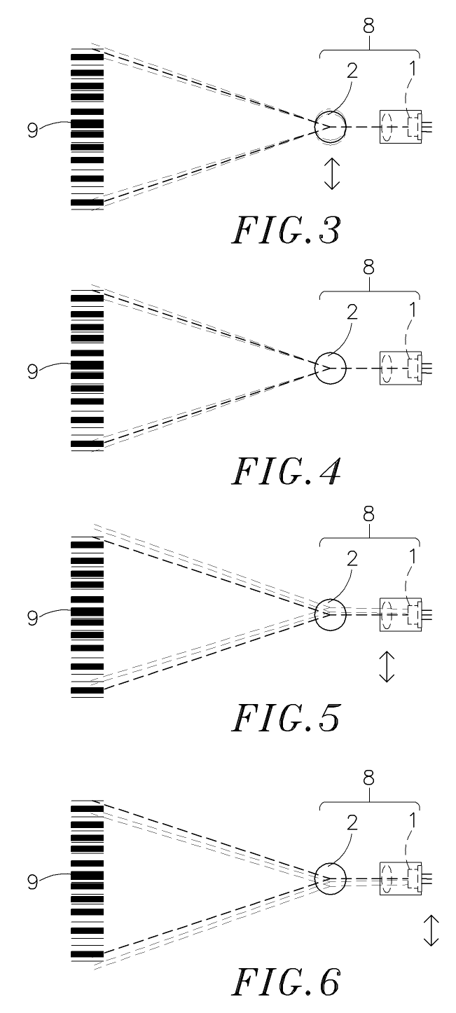

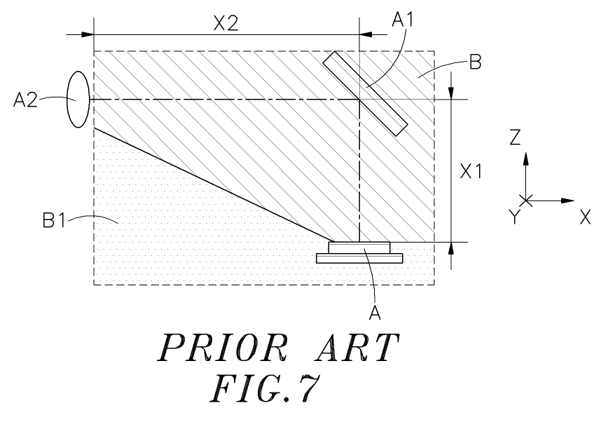

Referring to FIGS. 3˜6, the light emitting device of the light source 1 can be a laser diode. During operation, the light source 1 emits a laser light beam toward the standing cylinder lens 2, which expands the laser light beam into a line-shaped laser light beam that falls upon the barcode 9 of the product to be scanned. Further, the optical system is incorporated with a slight oscillation means 8 that reciprocates the laser light beam projected by the light source 1 in a micro-scale oscillation manner, enhancing uniformity of the projected line-shaped laser light beam. The slight oscillation means 8 causes the standing cylinder lens 2 to move back and forth in a micro-scale manner in direction perpendicular to the light path of the laser light beam that passes through the standing cylinder lens 2. Alternatively, slight oscillation of the standing cylinder lens 2 can be achieved by means of changing the crystal structure of the standing cylinder lens 2 like the functioning of a quartz oscillator. Further, the slight oscillation means 8 can be arranged to have the proximal side of the light source 1 that faces the standing cylinder lens 2 be moved back and forth in direction perpendicular to the light path. Alternatively, the slight oscillation means 8 can be arranged to have the distal side of the light source 1 remote from the standing cylinder lens 2 be moved back and forth in direction perpendicular to the light path. The functioning of the slight oscillation means 8 enhances uniformity of the light projected by the light source 1, avoiding image noise and improving barcode 9 reading reliability.

Further, the aforesaid slight oscillation means 8 can be to support the standing cylinder lens 2 on an elastic post for causing the desired slight oscillation in direction perpendicular to the light path of the light projected by the light source 1. Further, the slight oscillation means 8 can be any oscillator capable of slightly reciprocating the light source 1 or the standing cylinder lens 2 in direction perpendicular to the light projected by the light source.

Further, the light emitting device of the light source 1 can be a light emitting diode, a laser diode, or any suitable device capable of emitting light for illuminating a barcode for scanning.

As stated above, the invention provides an optical system for use in a barcode scanner that has the following advantages:

1. The invention utilizes a focusing lens to focus the image of the light reflected by the barcode to be scanned, and a slanted arranged reflector to reflect the focused image onto a liner sensor array. Further, the light path of the focusing lens is overlapped with the light path of the reflector. By means of the characteristic of this light path overlap region, the size of the barcode scanner is greatly reduced.

2. The invention uses a slight oscillation means to cause reciprocation of the light beam emitted by the light source in direction perpendicular to the light path, thereby enhancing uniformity of the projected light beam and improving barcode reading reliability.

A prototype of optical system for barcode scanner has been constructed with the features of FIGS. 1˜6. The optical system for barcode scanner functions smoothly to provide all of the features discussed earlier.

Although particular embodiments of the invention have been described in detail for purposes of illustration, various modifications and enhancements may be made without departing from the spirit and scope of the invention.

What the invention claimed is:

1. An optical system used in a barcode scanner for reading a barcode of a product, the optical system comprising a light source, a standing cylinder lens, a focusing lens, reflector means and a linear sensor array arranged on one same plane, wherein:
said light source is adapted to emit a light beam through said standing cylinder lens onto a barcode of a product;
said focusing lens is adapted to focus an image reflected by the barcode of the product, upon which the light beam emitted by said light source through said standing cylinder lens onto said reflector;
said reflector means is adapted to reflect the image focused by said focusing lens onto said linear sensor array for reading;
the light path of the image focused by said focusing lens onto said reflector and the light path of the image reflected by said reflector onto said linear sensor array are overlapped, forming a light path overlap region.

2. The optical system as claimed in claim 1, wherein said light source is comprised of a laser diode and a collimator.

3. The optical system as claimed in claim 1, wherein said light source is comprised of a light emitting diode and a collimator.

4. The optical system as claimed in claim 1, which said reflector means is a slanted arranged reflector.

5. The optical system as claimed in claim 1, which said reflector means comprises a plurality of reflectors arranged on said same plane.

6. The optical system as claimed in claim 1, further comprising slight oscillation means adapted to slightly oscillate the light beam emitted by said light source in direction perpendicular to the light path of said light source.

7. The optical system as claimed in claim 1, wherein said slight oscillation means is adapted to slightly oscillate said light source.

8. The optical system as claimed in claim 1, wherein said slight oscillation means is adapted to slightly oscillate said standing cylinder lens.

9. The optical system as claimed in claim 8, wherein said slight oscillation means is adapted to slightly oscillate said standing cylinder lens by means of applying different voltages to said standing cylinder lens to cause said standing cylinder lens to change a crystal structure thereof.
